(19) United States
(12) Patent Application Publication
(10) Pub. No.: US 2019/0064175 A1
(43) Pub. Date: Feb. 28, 2019

(54) METHODS FOR FABRICATING THREE-DIMENSIONAL LIPID STRUCTURE ARRAYS AND THREE-DIMENSIONAL LIPID STRUCTURE ARRAYS FABRICATED BY THE SAME

(71) Applicant: KOREA INSTITUTE OF SCIENCE AND TECHNOLOGY, Seoul (KR)

(72) Inventors: Tae Song Kim, Seoul (KR); Rhokyun Kwak, Seoul (KR); Won Bae Han, Seoul (KR)

(73) Assignee: KOREA INSTITUTE OF SCIENCE AND TECHNOLOGY, Seoul (KR)

(21) Appl. No.: 15/954,858

(22) Filed: Apr. 17, 2018

(30) Foreign Application Priority Data
Aug. 31, 2017 (KR) 10-2017-0110796

(51) Int. Cl. G01N 33/68

(52) U.S. Cl. CPC G01N 33/6842; G01N 2570/00

(57) ABSTRACT

Disclosed area methods for fabricating three-dimensional artificial lipid biomembrane structure arrays with sufficient reaction area and high stability on a substrate in a simpler and easier manner. The methods use constituent lipids of real cell membranes and a plurality of microwells formed on a substrate. The methods can more effectively provide biomimetic three-dimensional lipid membrane structure arrays that possess structural and/or functional properties of cell membranes.

### CROSS-REFERENCE TO RELATED APPLICATIONS

This application claims priority under 35 U.S.C. § 119 to Korean Patent Application No. 10-2017-0110796 filed on Aug. 31, 2017 in the Korean Intellectual Property Office, the disclosure of which is incorporated herein by reference in its entirety.

### BACKGROUND OF THE INVENTION

### 1. Field of the Invention

The present invention relates to methods for fabricating three-dimensional (3D) artificial lipid structure arrays with a size of several micrometers to several tens of micrometers. Particularly, the present invention relates to biomimetic three-dimensional structure arrays that possess structural and/or functional properties of cell membranes and have the ability to sense biomaterials or biosignals. More specifically, the present invention relates to methods for fabricating arrays of three-dimensional lipid biomembrane structures at particular positions on a solid substrate in a simpler and easier manner, and arrays of three-dimensional lipid biomembrane structures that are fabricated by the methods and are suitable for use as devices.

### 2. Description of the Related Art

Cells have functions of sensing and regulating changes outside cells and maintaining vital phenomena such as intracellular and extracellular communication. Cells consist of a ˜5-10 nm thick lipid bilayer (i.e. a unilamellar membrane) as an amphiphilic membrane and various membrane proteins, including ion channel proteins. The amphiphilic membrane plays a critical role in cellular functions. The membrane proteins immobilized onto the cell surface are difficult to biochemically analyze by standard methods optimized for general water-soluble proteins due to the presence of the hydrophobic region in the central portion of the lipid bilayer despite recent rapid and qualitative advances in various branches of biotechnology.

Analysis platforms consisting of artificial lipid structures need to be developed to fabricate a novel concept of biosensor that analyzes membrane proteins or uses membrane proteins. Recent research efforts have focused on the development of lipid bilayers mimicking the function and structure of real cells and the application of such lipid bilayers to biosensor devices by using 1) supported lipid bilayers (SUPLBs) consisting of an artificial lipid membrane and membrane proteins, which is formed on the surface of a solid such as a Si wafer or glass substrate or 2) suspended lipid bilayers (SUSLBs) in which micro- or nano-sized pores are formed as empty spaces without a supporting surface and a free-standing lipid membrane bilayer is formed on the empty spaces to form spaces on and under the membrane.

When supported lipid bilayers (SUPLBs) are used, the bilayers are placed on the surface of a solid or are located on a cushiony polymer material. The solid or polymer material suffers from difficulty in substantially having the bilayers reconstituted with membrane proteins in proper position depending on the size of membrane proteins and sometimes deteriorates the stability of membrane proteins. In contrast, suspended lipid bilayers (SUSLBs) maintain their bilayer structure with intracellular and extracellular spaces and are thus very similar to the structure of real cells. Accordingly, SUSLBs are optimal structures for performing model experiments mimicking various functions of cell membranes. The only disadvantage of SUSLBs is poor membrane stability. For this reason, attempts to enhance the structural stability of SUSLBs have been made by reducing the size of the pores to several micrometers to several hundreds of nanometers to minimize the area of the bilayers as small as possible or sandwiching a suitable porous polymer material (such as a hydrogel) between the spaces on and under the bilayers to reinforce the bilayers. SUPLBs and SUSLBs have a common feature in that planar lipid membranes are formed. Cells are usually unshaped, such as globular or curved, rather than planar.

In recent years, attempts have been made to construct three-dimensional globular lipid structures rather than planar ones. Takeuchi et al. at the University of Tokyo fabricated domed and vesicular lipid structure arrays using 1 μm deep wells formed on the surface of ITO glass. However, the domed structures are leaky due to their incomplete sealing and the vesicular structures are likely to be detached from the substrate. Further, Wostein' s group at the Pennsylvania State University succeeded in producing 10-70 μm-sized GUVs by transferring a lipid pattern to the surface of an ITO glass substrate using a hydrogel stamp, followed by electroforming in the presence of an electric field. These lipid structures are connectively attached to the ITO surface but are likely to be detached when the frequency of the AC electric field is reduced. That is, the construction of three-dimensional artificial biomembrane structures with sufficient reaction area and high stability in the direction perpendicular to a certain substrate using constituent lipids of cell membranes is essential to mimic the ability of cell membranes to sense biosignaling, which is an important function of cell membranes. However, three-dimensional artificial biomembrane structures immobilized onto a certain substrate have not been developed so far. Further, three-dimensional artificial biomembrane structures should be immobilized at desired positions and require perfect sealing while maintaining sufficient spaces from a substrate. However, none of these requirements are met.

Under these circumstances, Korean Patent No. 10-1608039 entitled “method for producing tubular lipid membrane, tubular lipid membrane produced by the method and biomembrane device including the tubular lipid membrane”, which was issued to the present applicant on Apr. 11, 2016. The method includes: placing porous templates having holes on one side of a support, dropping a lipid solution onto the porous templates, and drying the porous templates to prepare a substrate including lipid film layers; bonding the lipid film layers of the substrate to an adhesive layer formed on one side of the support and removing the support; and hydrating the lipid film layers by adding a buffer to the lipid film layers of the substrate, to form tubular structures extending from the lipid film layers through the holes of the porous templates. However, the use of the porous templates having holes in addition to the support is troublesome and the bonding of the lipid film layers of the substrate to the adhesive layer makes the production process complicated.

### SUMMARY OF THE INVENTION

The present invention has been made in an effort to solve the above problems, and it is an object of the present invention to provide methods for fabricating three-dimensional artificial lipid biomembrane structure arrays with sufficient reaction area and high stability on a substrate in a simpler and easier manner. That is, the present invention is aimed at providing methods for fabricating three-dimensional lipid membrane structure arrays capable of more effectively sensing biosignals on a substrate in a simpler and easier manner than conventional free bilayer vesicles or planar bilayer structure arrays using substrates.

The present invention is also aimed at providing biosignal sensing devices that possess structural and/or functional properties of cell membranes and can mimic the ability of cell membranes to sense biosignals, which is an important function of cell membranes.

The present invention is also aimed at fabricating a three-dimensional globular artificial unilamellar lipid membrane structure array attached to a substrate in a simple and easy manner.

One embodiment of the present invention provides a method for fabricating a lipid structure array, including: preparing an array of a plurality of microwells formed on a substrate; introducing a lipid solution into the microwells and drying the microwell array to form lipid layers; and hydrating the lipid layers by dropping a buffer solution onto the lipid layers formed in the microwells, to form three-dimensional structures extending from the lipid layers on the microwells.

The lipid solution may be a solution including trichloroethylene.

The lipids are preferably present at a concentration ranging from 1 to 50 mM in the lipid solution.

The formation of lipid layers includes dropping a lipid solution onto the surface of the microwell array and rotating the microwell array such that the lipid solution is introduced into the microwells and drying the microwell array.

The introduction of the lipid solution includes treating the surface of the microwell array with an oxygen plasma to hydrophilize the substrate surface and dropping the lipid solution on the hydrophilized substrate surface.

The introduction of the lipid solution preferably includes introducing the lipid solution into the microwells and removing the lipid solution remaining outside the microwells by suction.

The microwell array is dried at a temperature ranging from −10 to −80° C.

The microwell array is dried at a pressure ranging from 1 to 10 mTorr for 5 to 20 hours.

The buffer solution is preferably distilled (DI) water.

The microwells may have a diameter ranging from 1 to 20μm.

Preferably, the microwells have an aspect ratio (depth/diameter) ranging from 0.2 to 10.0.

The plurality of microwells may have a pitch ranging from 10 to 100 μm.

The three-dimensional structures may be tubular structures.

The three-dimensional structures may have an areal strain εₐ of at least 0.5, as calculated by Equation 1:

εₐ=(Aₑ−Aᵤ)/Aᵤ=Aₑ/Aᵤ−1 (1)

where Aᵤ represents the unit area of the substrate and is determined from the repeated pattern of the microwells, and Aₑ represents the estimated lipid deposit of the lipid layers formed in the microwells and is estimated from a fluorescence image of the lipid structures on the microwells.

A further embodiment of the present invention provides a lipid structure array including: an array of a plurality of microwells formed on a substrate; lipid layers formed by introducing a lipid solution into the microwells and drying the microwell array; and three-dimensional structures extending or produced from the lipid layers on the microwells.

The microwells may have a diameter ranging from 1 to 20 μm.

Preferably, the microwells have an aspect ratio (depth/diameter) ranging from 0.2 to 10.0.

The plurality of microwells may have a pitch ranging from 10 to 100 μm.

The three-dimensional structures may be tubular structures.

Another embodiment of the present invention provides a method for fabricating a lipid structure array, including: preparing an array of a plurality of microwells formed on a substrate; introducing a lipid solution into the microwells and drying the lipid microwell array to form lipid layers; and hydrating the lipid layers by dropping a buffer onto the lipid layers formed in the microwells in the presence of an electric field applied perpendicular to the microwells, to form three-dimensional structures extending from the lipid layers on the microwells.

The three-dimensional structures may be globular structures consisting of unilamellar bilayers.

Specific details of other embodiments are included in the following description and accompanying drawing.

According to the methods of the present invention, a plurality of microwells formed on a substrate are used to produce three-dimensional long artificial lipid biomembrane structures on the substrate structures in a simpler and easier manner.

In addition, the biomimetic arrays of the present invention consist of three-dimensional lipid membranes, possess structural and/or functional properties of cell membranes, and can mimic the ability of cell membranes to sense biosignaling, which is an important function of cell membranes.

Furthermore, according to the method of the present invention, a buffer is dropped onto lipid layers formed in microwells to hydrate the lipid layers in the presence of an electric field applied perpendicular to the microwells, enabling the production of three-dimensional globular artificial unilamellar bilayer structures on a substrate in a simple and easy manner.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIGS. 1A, 1B and 1C illustrate methods for fabricating a lipid structure array according to preferred embodiments of the present invention;

FIG. 2 illustrates exemplary cross-sectional views for explaining the preparation of a microwell array in a method of the present invention;

FIG. 3 shows exemplary scanning electron microscopy images for explaining the formation of microwells of a microwell array in a method of the present invention;

FIG. 4 illustrates exemplary cross-sectional views for explaining the formation of lipid layers in a method of the present invention;

FIG. 5 is an exemplary fluorescence microscopy image for explaining the formation of lipid layers in microwells of a microwell array in a method of the present invention;

FIG. 6 is an exemplary cross-sectional view for explaining the diameter and aspect ratio (depth/diameter) of microwells formed in a method of the present invention and the pitch between the microwells;

FIG. 7 shows exemplary fluorescence microscopy images of three-dimensional structures and self-spreading bilayers formed on microwells arranged at different pitches and an exemplary atomic force microscopy image of self-spreading lipid bilayers;

FIG. 8 shows exemplary confocal fluorescence microscopy images of three-dimensional structures formed by a method of the present invention;

FIG. 9 exemplarily shows unit areas on substrates, which were determined from repeated patterns of microwells, and estimated lipid deposits of lipid layers formed in the microwells, which were estimated from fluorescence images of lipid structures, to calculate an areal strain inducing the formation of three-dimensional structures in a method of the present invention;

FIG. 10 exemplarily shows relation of unit areas and estimated lipid deposits to form both three-dimensional structures and self-spreading bilayers by a method of the present invention;

FIG. 11 is an exemplary confocal fluorescence microscopy image showing globular (unilamellar bilayer) structures formed by the application of an electric field to lipid layers in a method of the present invention; and

FIG. 12 is an exemplary schematic diagram for explaining the formation of three-dimensional globular structures by the application of an electric field to lipid layers in a method of the present invention.

### DETAILED DESCRIPTION OF THE INVENTION

As the present invention allows for various changes and numerous embodiments, particular embodiments will be illustrated in drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the description of the present invention, detailed explanations of related art are omitted when it is deemed that they may unnecessarily obscure the essence of the present invention.

The terminology used herein is for the purpose of describing particular embodiments only and is not intended to be limiting. As used herein, the singular forms intend plural forms as well, unless the context clearly indicates otherwise. In the present application, it is to be understood that the terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

While such terms as “first” and “second,” etc., may be used to describe various components, such components must not be limited to the above terms. The above terms are used only to distinguish one component from another.

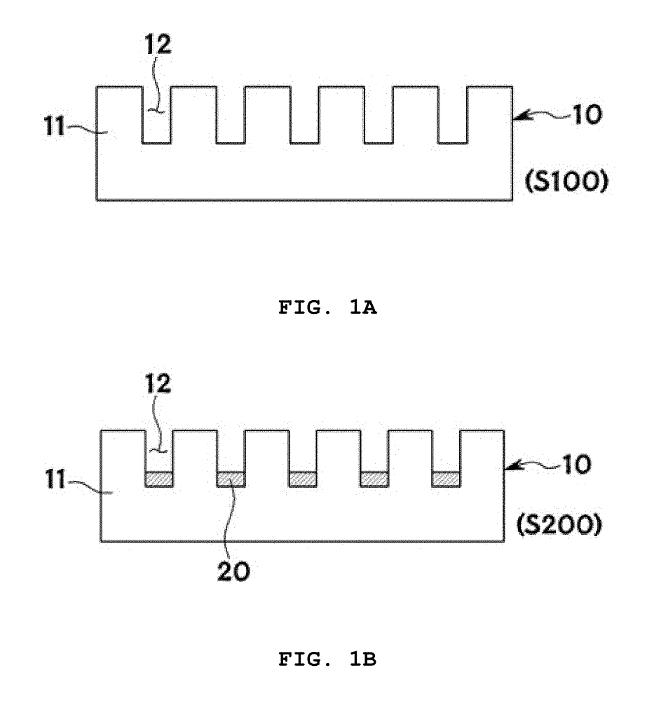

FIGS. 1A, 1B and 1C illustrate methods for fabricating a lipid structure array according to preferred embodiments of the present invention.

As illustrated in this figure, the method includes preparing a microwell array 10 (S100), forming lipid layers 20 (S200), and forming three-dimensional structures 40 (S300).

In S100, a microwell array 10 is prepared. Specifically, an array of a plurality of microwells 12 is prepared on a substrate 11. The substrate serves as a support on which lipid structures are to be formed. The substrate 11 is not particularly limited and may be any of those known in the art. For example, the substrate 11 may be a silicon plate or substrate. The microwells 12 are spaces where a lipid solution is to be filled to form lipid layers 20. The shape of the microwells 12 is not particularly limited. For example, the microwells 12 may have a rectangular shape in cross section or may have a cylindrical shape. The plurality of microwells 12 may be arranged at regular pitches on the substrate 11.

In S200, a lipid solution is introduced into the microwells 12 and the microwell array is then dried to form lipid layers 20. The lipid solution may be a solution of lipids in a solvent. In the method of the present invention, lipid structures are formed using lipids. There is no particular restriction on the method for introducing the lipid solution and drying the microwell array. A more detailed description will be given about the lipid solution and the formation of the lipid layers 20 using the lipid solution.

In S300, three-dimensional structures 40 are formed. A buffer 30 is dropped onto the lipid layers 20 formed in the microwells to hydrate the lipid layers, resulting in the formation of three-dimensional structures 40 extending from the lipid layers 20 on the microwells. The buffer 30 is added to hydrate the lipid layers 20. The buffer 30 is not particularly limited and may be, for example, phosphate buffered saline (PBS). The use of distilled (DI) water as the buffer is more preferred because DI water is the simplest solvent that can be used irrespective of pH, ion concentration, and other factors.

Lipids are basically amphiphilic molecules that possess both hydrophilic and hydrophobic groups. The hydrophilic heads are directed outward and the hydrophobic tails are directed inward to form bilayers. The dried lipid layers 20 consist of several lipid bilayers stacked on one another. When the buffer 30 is added to the lipid layers 20, water molecules are infiltrated into the interstices between the lipid bilayers to create an osmotic pressure, resulting in separation of the lipid layers.

The lipid layers can be hydrated to construct lipid structures by various processes known in the art. For example, globular lipid structures such as giant unilamellar vesicles (GUVs) may be constructed by coating a lipid film on a solid substrate such as glass and dropping a buffer onto the lipid film to hydrate the lipid film (“Giant Vesicles: Preparations and Applications”, ChemBioChem 2010, 11, 845-865., “Liposomes: Technologies and Analytical Applications”, Annu. Rev. Anal. Chem. 2008, 1, 801-832).

The method of the present invention uses lipids to produce artificial biomembrane structures on a substrate. Particularly, the use of a plurality of microwells formed on the substrate enables the formation of three-dimensional structures in a simpler and easier manner. The three-dimensional structures may be tubular structures.

Conventional thermal, electromechanical, biological, mechanical, and electrical methods for producing three-dimensional lipid structures have failed to produce artificial biomembrane structures immobilized onto substrates. The present inventors have succeeded in developing a method for producing tubular lipid membranes using porous templates having holes. However, this method is troublesome and complicated to carry out. As a result of intensive research efforts to solve the above problems, the present inventors have found that the use of a plurality of microwells enables the production of tubular or vesicular lipid membrane structures capable of more effectively sensing biosignals in a simpler and easier manner than planar bilayers using conventional substrates or vesicular liposomal bilayers floating in three-dimensional spaces, accomplishing the present invention.

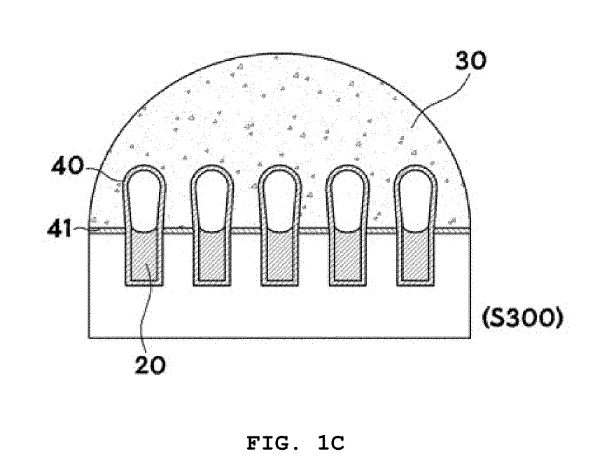

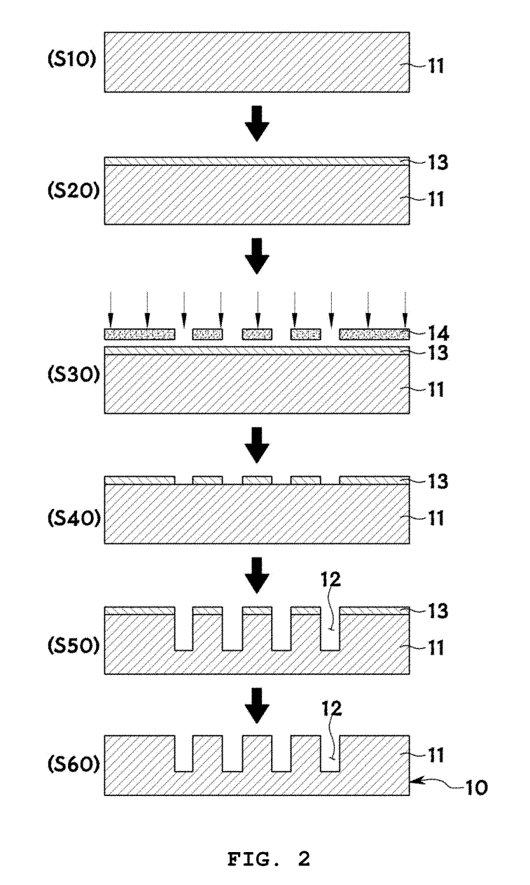

FIG. 2 illustrates exemplary cross-sectional views for explaining the preparation of a microwell array (S100) in the method of the present invention and FIG. 3 shows exemplary scanning electron microscopy images for explaining the formation of microwells of a microwell array in the method of the present invention (scale bar: 10 μm).

In S100, a microwell array 10 is prepared. Specifically, an array of a plurality of microwells 12 is prepared on a substrate 11. There is no particular restriction on the method for forming the plurality of microwells 12 on a substrate 11. A procedure for forming the microwells 12 is exemplarily illustrated in FIG. 2. First, a silicon substrate 11 is prepared (S10) and a photoresist is coated thereon to form a photoresist film 13 (S20). Then, a chromium mask 14 is placed on the photoresist film 13 and is exposed through lithography (S30). Subsequently, the photoresist film 13 is developed to form a pattern. The silicon is anisotropically etched by deep trench RIE (S40). Finally, the photoresist film 13 is removed with an oxygen plasma (S50).

The resulting microwells 12 may have various diameters, depths, and aspect ratios (depth/diameter), as shown in FIG. 3.

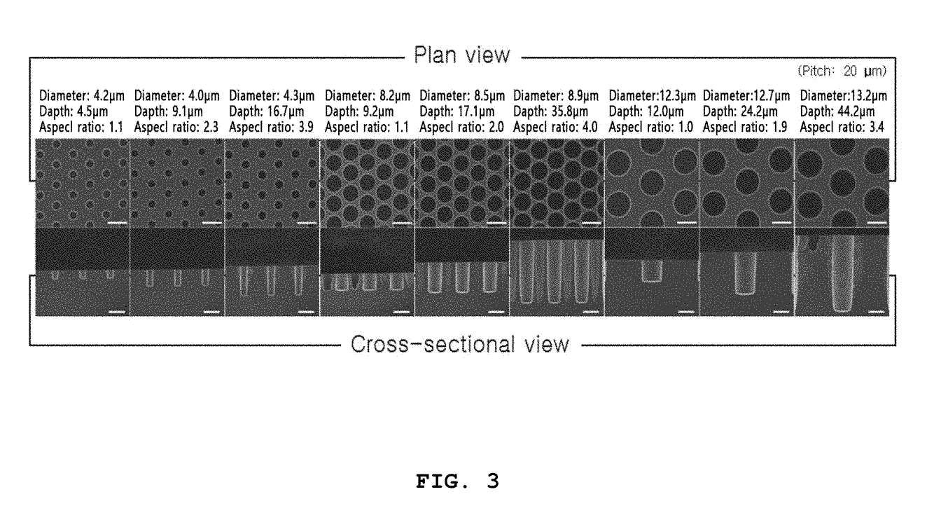

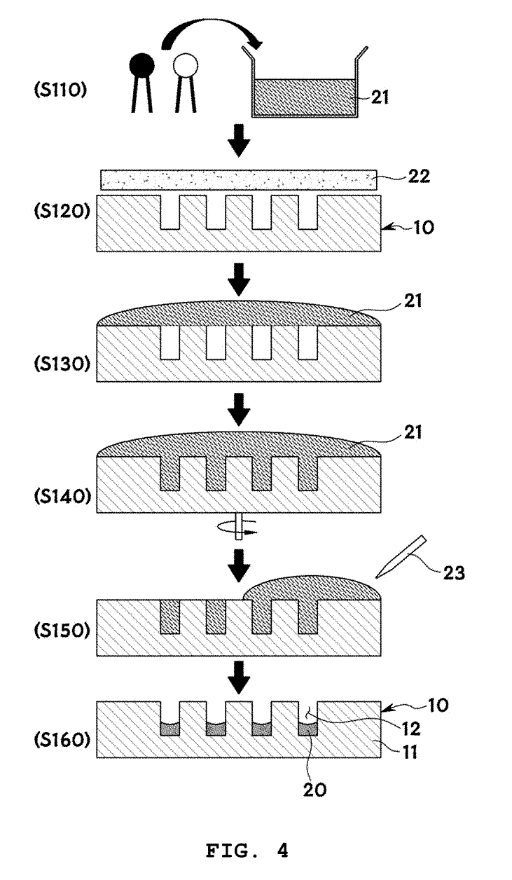

FIG. 4 illustrates exemplary cross-sectional views for explaining the formation of lipid layers 20 in the method of the present invention and FIG. 5 is an exemplary fluorescence microscopy image for explaining the formation of lipid layers 20 in the microwells 12 of the microwell array in the method of the present invention.

In S200, a lipid solution is introduced into the microwells 12 and the microwell array is then dried to form lipid layers 20. There is no particular restriction on the method for introducing the lipid solution into the microwells 12 and drying the microwell array.

For example, first, a lipid solution 21 is prepared (S110). The lipid solution 21 is a solution including lipids. The concentration of lipids in the lipid solution 21 is preferably in the range of 1 to 50 mM. Any organic solvent such as chloroform or methanol that can dissolve lipids may be used without particular limitation to prepare the lipid solution. The solvent is most preferably trichloroethylene that has an appropriate contact angle for the silicon substrate. The use of trichloroethylene allows for pinning of the lipid solution into the wells, enabling selective coating of the wells with the lipid solution. Other solvents are not suitable for selective coating.

The lipid solution 21 may further include fluorescently labeled lipids for subsequent identification by fluorescence microscopy. The fluorescently labeled lipids may lipids whose heads are attached with a green fluorescent material and/or a red fluorescent material. For example, the lipids may be composed of 1,2-dioleoyl-sn-glycero-3-phosphoethanolamine (DOPE), the green fluorescent material may be NBD, and the red fluorescent material may be rhodamine B. For example, the fluorescently labeled lipids may be composed of 1,2-dioleoyl-sn-glycero-3-phosphoethanolamine-N-(7-nitro-2-1,3-benzoxadiazol-4-yl) (NBD-PE) and/or 1,2-dioleoyl-sn-glycero-3-phosphoethanolamine-N-(lissamine rhodamine B sulfonyl (Liss Rhod PE) and may be used in an amount ranging from 0.1 to 10 mol %, preferably from 0.3 to 1.0 mol %, based on the total moles of all lipids in the lipid solution 21.

The surface of the microwell array 10 is treated with an oxygen plasma 22 to hydrophilize the substrate surface (S120). There is no restriction on the method for hydrophilizing the substrate surface. For example, the substrate surface may be made hydrophilic by chemical cleaning such as piranha cleaning. Particularly, oxygen plasma treatment is more effective in hydrophilizing the substrate surface in a safer and simpler manner.

Then, the lipid solution 21 is dropped on the hydrophilic substrate surface (S130).

Subsequently, the microwell array is rotated such that the lipid solution 21 is introduced into the microwells 12 of the microwell array (S140). The microwell array is specially designed to be rotatable. As a result of this rotation, the lipid solution 21 is introduced into the microwells 12 of the microwell array. For example, the lipid solution 21 may be agitated by spinning at low speed. The rotation of the microwell array 10 at a speed as low as 100 rpm enables more effective introduction of the lipid solution 21 into the microwells 12.

The method may include removing the lipid solution remaining outside the microwells by suction using a pump 23 after introduction of the lipid solution 21 into the microwells 12 of the microwell array 10 (S150). For example, a syringe pump may be used to suck the remaining lipid solution 21.

Then, the microwell array 10 is dried to form lipid layers 20 (S160). There is no particular restriction on the method for drying the microwell array. The microwell array 10 is preferably dried at a temperature ranging from −10 to −80° C. More preferably, the microwell array is dried at a pressure ranging from 1 to 10 mTorr for 5 to 20 hours. For example, the microwell array 10 may be dried in a freeze dryer. It is preferred that the drying temperature is lower than the transition temperature of DOPC (lipids) (−17° C.). The drying temperature is more preferably in the range of −20° C. to −70° C. Within this range, the lipids are protected from denaturing. The most effective drying conditions are 5 mTorr and at least 6 hours where trichloroethylene molecules used to dissolve the lipids can be completely evaporated.

FIG. 5 is a fluorescence microscopy image of the substrate selectively coated with the lipids (Scale bar: 30 μm). The line profile of the lipid-coated substrate reveals that an intensity of 0 (zero) was observed in areas other than the microwells. This observation demonstrates that the lipids were selectively coated in the microwells.

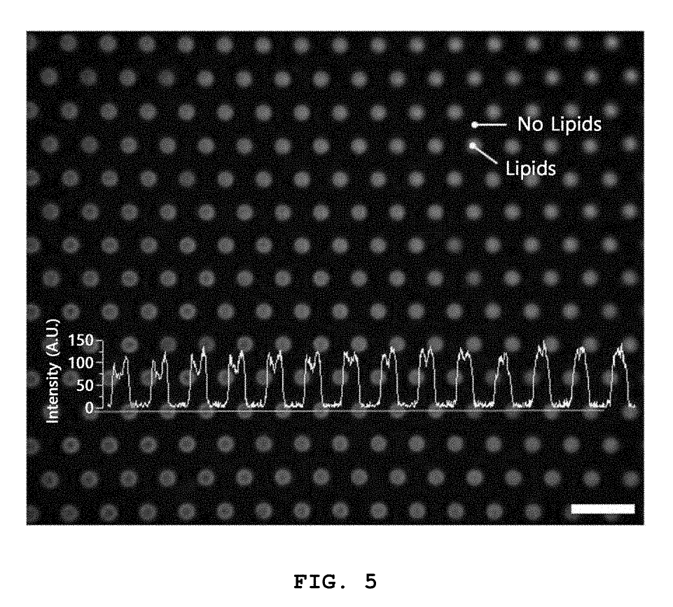

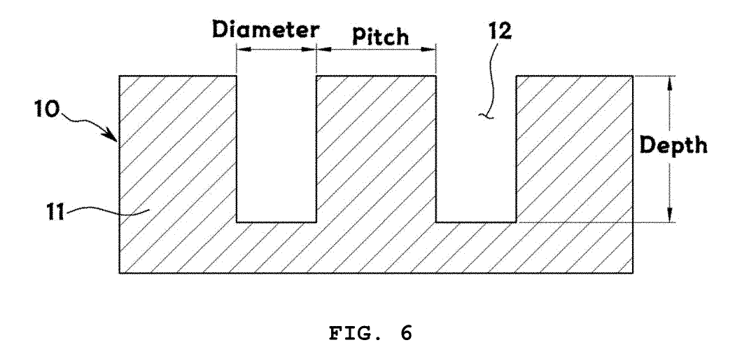

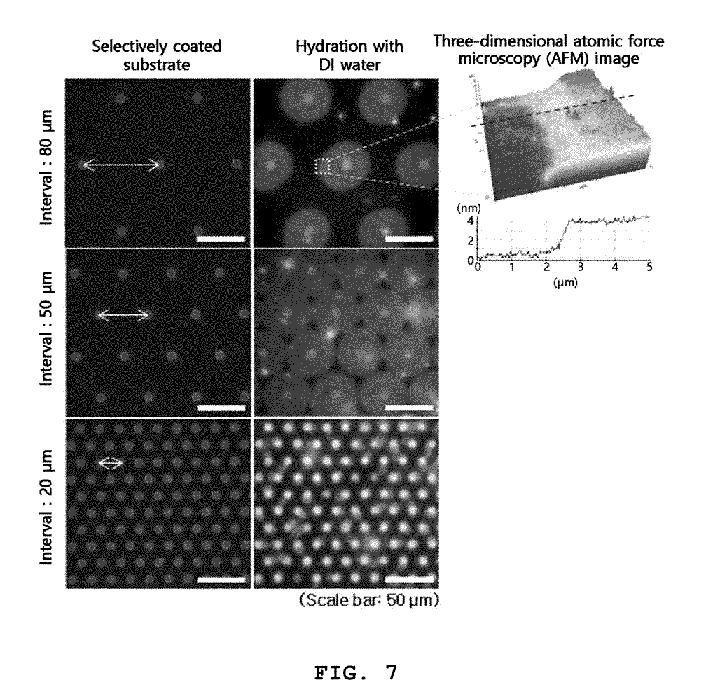

FIG. 6 is an exemplary cross-sectional view for explaining the diameter and aspect ratio (depth/diameter) of microwells formed in the method of the present invention and the pitch between the microwells. FIG. 7 shows exemplary fluorescence microscopy images of three-dimensional structures and self-spreading bilayers formed on microwells arranged at different pitches and an exemplary atomic force microscopy image of self-spreading lipid bilayers. FIG. 8 shows exemplary confocal fluorescence microscopy images of three-dimensional structures formed by the method of the present invention.

The present inventors have conducted research through numerous experiments to more effectively form three-dimensional structures using microwells formed on a substrate by varying the diameter, depth, and aspect ratio (depth/diameter) of the microwells, the pitches between the microwells, and the concentration of lipids in a lipid solution.

FIG. 7 shows lipid structures formed on arrays of microwells arranged at different pitches of 80 μm, 50 μm, and 20 μm by using a lipid solution having a concentration of 10 mM.

As shown in this figure, when the substrates selectively coated with the lipid solution were hydrated with distilled (DI) water, lipid bilayers spread outward from the lipid-coated microwells. This phenomenon is called “self-spreading”. Two-dimensional structures were formed on arrays of microwells arranged at pitches of 80 μm and 50 μm by self-spreading of the lipids (see the top and middle images of FIG. 7, respectively). Three-dimensional atomic force microscopy (AFM) of the top right image of FIG. 7 reveals that the planar lipid structures were ˜4 nm thick, demonstrating self-spreading of the lipid bilayers.

Three-dimensional structures and self-spreading bilayers were formed on an array of microwells arranged at pitches of 20 μm (see the bottom images of FIG. 7). In the bottom right image of FIG. 7, self-spreading was not clearly observed on the surface of the substrate due to the contrast of the fluorescence intensities but the self-spreading bilayers covering the entire area of the substrate surface were observed together with the three-dimensional structures. When the pitches between the microwells were 20 μm, the self-spreading bilayers met and even covered the entire area of the substrate surface, similarly to when the pitches between the microwells were 50 μm. After the entire area of the substrate surface was covered with the lipids, self-spreading did not occur any more. Thereafter, stress was applied to the lipids that remained sufficiently in the microwells, and the lipid membranes began to swell. This swelling appears to lead to the formation of the three-dimensional structures.

These results concluded that when a lipid solution having the same concentration is used, three-dimensional lipid structures can be formed on microwells arranged at sufficiently small pitches. According to the method of the present invention, it is preferred that the pitches between the plurality of microwells are small so long as the structures formed on the microwells do not interfere with one another. It is particularly preferred that the pitches between the plurality of microwells is in the range of 10 to 100 μm, 10 to 90 μm, 20 to 90 μm, 20 to 80 μm, 10 to 60 μm or 10 to 50 μm.

When the pitches between the plurality of microwells are large, three-dimensional structures can be formed by controlling the concentration of the lipid solution and/or the diameter and aspect ratio (depth/diameter) of the microwells. FIG. 8 shows the lipid structures formed on the array of microwells arranged at pitches of 50 μm using the lipid solution having a concentration of 20 mM. The confocal fluorescence microscopy images of FIG. 8 reveal that the surface was covered with self-spreading bilayers and three-dimensional lipid structures were formed in and on the microwells. Specifically, when the pitches between the plurality of microwells were large, lipids were used at a high concentration to form three-dimensional lipid structures. It is preferred that the concentration of lipids in the lipid solution is in the range of 1 to 100 mM, 2 to 80 mM, 3 to 50 mM, 5 to 30 mM, 5 to 20 mM or 10 to 20 mM.

Thus, when three-dimensional lipid structures are formed using microwells on a substrate, the concentration of lipids, the diameter and aspect ratio of the microwells, and the pitches between the microwells are considered important factors. Particularly, it was found that the diameter of the microwells is more preferably in the range of 1 to 20 μm, 2 to 18 μm, 3 to 16 μm, 4 to 15 μm, 6 to 14 μm, or 8 to 12 μm. It was also found that the aspect ratio (depth/diameter) of the microwells is in the range of 0.2 to 10.0, 0.2 to 8.0, 0.2 to 6.0, 0.2 to 5.0, 0.3 to 4.0, or 0.4 to 4.0.

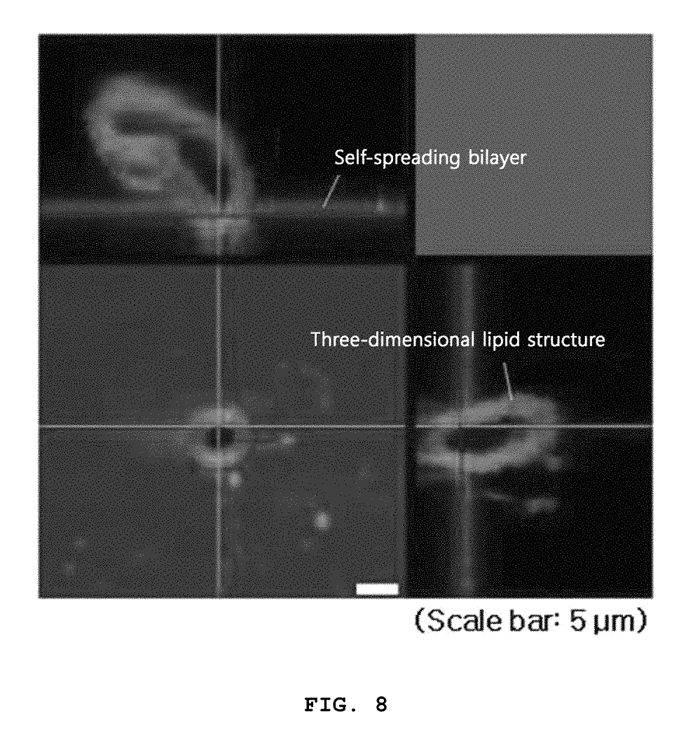

FIG. 9 is an exemplary diagram for explaining an areal strain inducing the formation of three-dimensional structures in the method of the present invention and schematically shows a plurality of microwells on a substrate and self-spreading around one of the microwells.

According to the method of the present invention, three-dimensional lipid structures can be formed using microwells on a substrate by controlling the concentration of lipids, the diameter and aspect ratio of the microwells, and the pitches between the microwells, as described above.

The present inventors have tried to standardize or normalize the conditions for the formation of three-dimensional lipid structures, including the concentration of lipids, the diameter and aspect ratio of microwells, and the pitches between the microwells, and as a result, have succeeded in deriving the following equation 1:

εₐ=(Aₑ−Aᵤ)/Aᵤ=Aₑ/Aᵤ−1 (1)

where εₐ represents the areal strain, Aᵤ represents the unit area of the substrate and is determined from the repeated pattern of the microwells, and Aₑ represents the estimated lipid deposit of the lipid layers formed in the microwells and is estimated from a fluorescence image of the lipid structures on the microwells.

The areal strain is the difference between the unit area Aᵤ and the estimated lipid deposit Aₑ and refers to stress generated in the microwells. When the areal strain is 0 (zero), it means that the self-spreading lipid bilayers cover the entire area of the pattern. When the areal strain is greater than 0 (zero), it means that the self-spreading lipid bilayers completely cover the entire area of the substrate surface and some of the lipids remain in the microwells. The remaining lipids are used to form the three-dimensional structures.

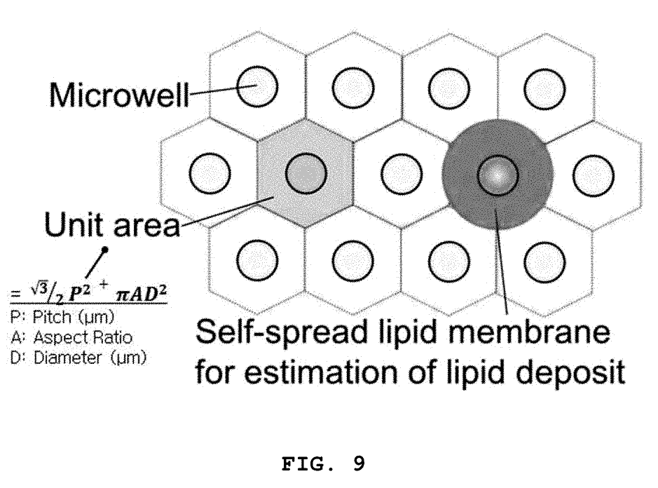

FIG. 10 exemplarily shows relation of unit areas and estimated lipid deposits to form both three-dimensional structures and self-spreading bilayers by a method of the present invention.

In the graph of FIG. 10, the data points are experimental results on the formation of three-dimensional lipid structures. For example, the data point indicated by the black dashed circle in FIG. 10 means that three-dimensional structures were formed by coating of 10 mM lipids on a substrate consisting of microwells having a diameter of 12 μm, an aspect ratio of 2, and a pitch of 50 μm.

The straight line shows results obtained by fitting experimental data points on the minimum lipid deposits of the three-dimensional lipid structures in the unit areas. The slope of the straight line was 1.52, indicating that the three-dimensional lipid structures began to form from when the areal strain was ˜0.5 or above.

These results lead to the conclusion that the areal strain of the three-dimensional structures is preferably at least 0.5, as calculated by Equation 1.

A further embodiment of the present invention provides a lipid structure array fabricated by the method. The lipid structure array of the present invention includes an array of a plurality of microwells formed on a substrate, lipid layers formed by (selectively) introducing a lipid solution into the microwells and drying the microwell array, and three-dimensional structures extending or produced from the lipid layers on the microwells.

The three-dimensional structures are formed by hydrating the lipids in the plurality of microwells formed on the substrate. The three-dimensional structures are more preferably tubular structures. The biomimetic three-dimensional structure array of the present invention possesses structural and/or functional properties of cell membranes and can mimic the ability of cell membranes to sense biosignaling, which is an important function of cell membranes.

Preferably, the microwells have a diameter ranging from 1 to 20 μm and an aspect ratio (depth/diameter) ranging from 0.2 to 10.0. The pitches between the plurality of microwells may be in the range of 10 to 100 μm. The dimensions of the microwells are the same as those described above.

Another embodiment of the present invention provides a three-dimensional globular lipid structure array fabricated by applying an electric field to lipid layers. Tubular structures were formed on microwells of a substrate in the absence of an electric field whereas globular structures were formed in the presence of an electric field.

Thus, another embodiment of the present invention provides a method for fabricating a lipid structure array, including: preparing an array of a plurality of microwells formed on a substrate; introducing a lipid solution into the microwells and drying the lipid microwell array to form lipid layers; and hydrating the lipid layers by dropping a buffer onto the lipid layers formed in the microwells in the presence of an electric field applied perpendicular to the microwells, to form three-dimensional structures from the lipid layers on the microwells.

The preparation of the microwell array and the formation of the lipid layers are the same as those described above.

The method of the present invention is characterized in that an electric field is applied perpendicular to the microwells prior to, during or after dropping of a buffer onto the lipid layers formed in the microwells. The application of an electric field is not limited to a particular method or apparatus.

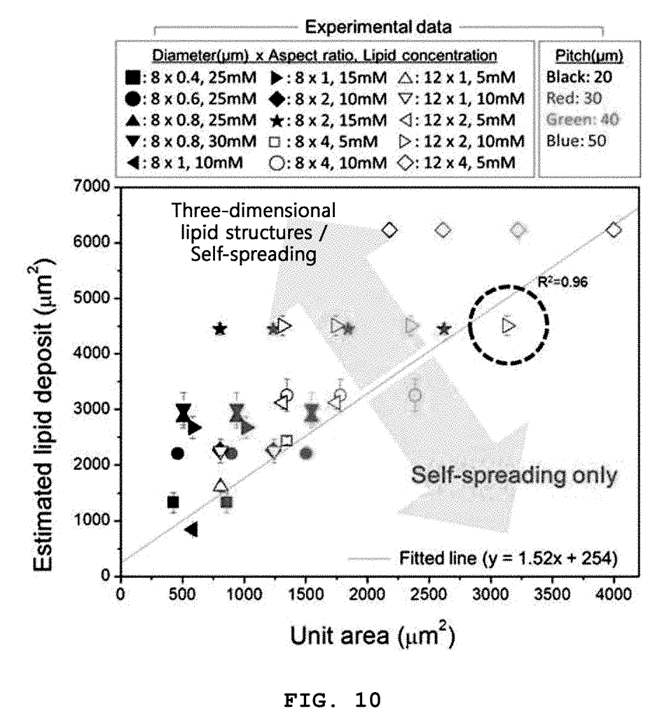

FIG. 11 is an exemplary confocal fluorescence microscopy image showing globular structures formed by the application of an electric field to lipid layers in the method of the present invention. As shown in this figure, the surface was covered with self-spreading bilayers and three-dimensional unilamellar lipid bilayer structures were formed in and on the microwells.

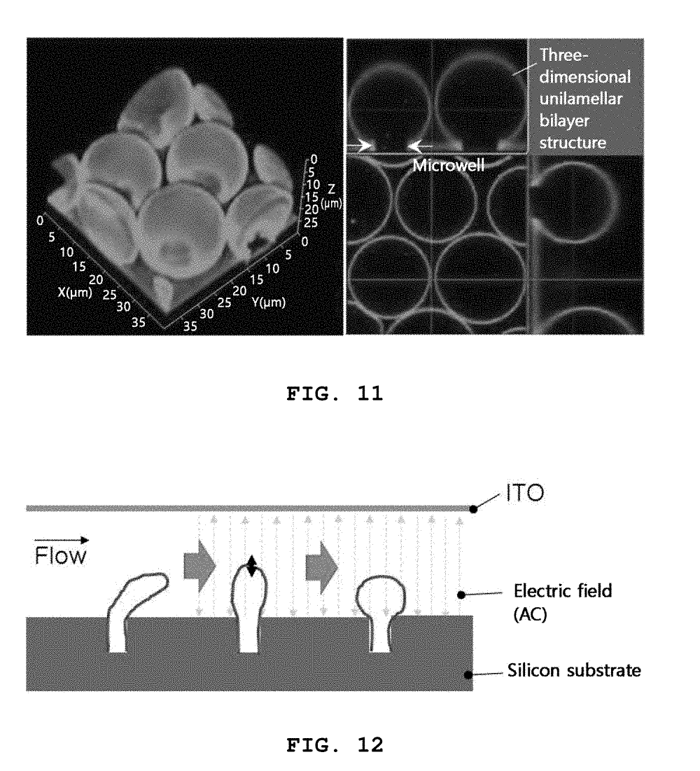

FIG. 12 is an exemplary schematic diagram for explaining the formation of three-dimensional globular structures by the application of an electric field to lipid layers in the method of the present invention. As shown in this figure, when an electric field was applied to lipid layers, the lipid membranes underwent repeated cycles of swelling and fusion to form a single larger unilamellar bilayer.

The heads and tails of lipids are asymmetric in size. Due to this asymmetry, three-dimensional lipid structures (generally, elongated tubular structures) are converted to the most stable structures with the passage of time. That is to say, the most stable structures are globular structures whose system energy, including curvature energy, is reduced to the smallest possible value. The application of an electric field allows the three-dimensional structures to be placed in a direction perpendicular to the electric field between ITO and the silicon substrate and causes the three-dimensional structures to be aligned along the direction of the electric field. This phenomenon appears to explain the formation of globular structures.

According to the method of the present invention, three-dimensional globular artificial biomembrane structures can be formed on a substrate in a simple and easy manner by dropping a buffer onto lipid layers formed in microwells in the presence of an electric field applied perpendicular to the microwells to hydrate the lipid layers.

A lipid structure array fabricated by the method of the present invention includes: an array of a plurality of microwells formed on a substrate; lipid layers formed by introducing a lipid solution into the microwells and drying the microwell array; and three-dimensional globular structures extending or produced from the lipid layers on the microwells.

While the invention has been particularly shown and described with reference to preferred embodiments thereof, it will be apparent to those skilled in the art that various modifications and variations are possible, without departing from the technical spirit and scope of the invention as defined by the appended claims.

What is claimed is:

1. A method for fabricating a lipid structure array, comprising: preparing an array of a plurality of microwells formed on a substrate; introducing a lipid solution into the microwells and drying the microwell array to form lipid layers; and hydrating the lipid layers by dropping a buffer solution onto the lipid layers formed in the microwells, to form three-dimensional structures produced from the lipid layers on the microwells.

2. The method according to claim 1, wherein the lipid solution is a solution comprising trichloroethylene.

3. The method according to claim 1, wherein the lipids are present at a concentration ranging from 1 to 50 mM in the lipid solution.

4. The method according to claim 1, wherein the formation of lipid layers comprises dropping a lipid solution onto the surface of the microwell array and rotating the microwell array such that the lipid solution is introduced into the microwells and drying the microwell array.

5. The method according to claim 4, wherein introduction of the lipid solution comprises treating the surface of the microwell array with an oxygen plasma to hydrophilize the substrate surface and dropping the lipid solution on the hydrophilized substrate surface.

6. The method according to claim 4, wherein the introduction of the lipid solution comprises introducing the lipid solution into the microwells and removing the lipid solution remaining outside the microwells by suction.

7. The method according to claim 4, wherein the microwell array is dried at a temperature ranging from −10 to −80° C.

8. The method according to claim 7, wherein the microwell array is dried at a pressure ranging from 1 to 10 mTorr for 5 to 20 hours.

9. The method according to claim 1, wherein the buffer solution is distilled (DI) water.

10. The method according to claim 1, wherein the microwells have a diameter ranging from 1 to 20 μm.

11. The method according to claim 1, wherein the microwells have an aspect ratio (depth/diameter) ranging from 0.2 to 10.0.

12. The method according to claim 1, wherein the plurality of microwells have a pitch ranging from 10 to 100 μm.

13. The method according to claim 1, wherein the three-dimensional structures are tubular structures.

14. The method according to claim 1, wherein the three-dimensional structures have an areal strain εₐ of at least 0.5, as calculated by Equation 1:
εₐ=(Aₑ−Aᵤ)/Aᵤ=Aₑ/Aᵤ−1 (1)
where Aᵤ represents the unit area of the substrate and is determined from the repeated pattern of the microwells, and Aₑ represents the estimated lipid deposit of the lipid layers formed in the microwells and is estimated from a fluorescence image of the lipid structures on the microwells.

15. A lipid structure array comprising: an array of a plurality of microwells formed on a substrate; lipid layers formed by introducing a lipid solution into the microwells and drying the microwell array; and three-dimensional structures produced from the lipid layers on the microwells.

16. The lipid structure array according to claim 15, wherein the microwells have a diameter ranging from 1 to 20μm.

17. The lipid structure array according to claim 15, wherein the microwells have an aspect ratio (depth/diameter) ranging from 0.2 to 10.0.

18. The lipid structure array according to claim 15, wherein the plurality of microwells have a pitch ranging from 10 to 100 μm.

19. The lipid structure array according to claim 15, wherein the three-dimensional structures are tubular structures.

20. A method for fabricating a lipid structure array, comprising: preparing an array of a plurality of microwells formed on a substrate; introducing a lipid solution into the microwells and drying the lipid microwell array to form lipid layers; and hydrating the lipid layers by dropping a buffer onto the lipid layers formed in the microwells in the presence of an electric field applied perpendicular to the microwells, to form three-dimensional structures produced from the lipid layers on the microwells.

21. The method according to claim 20, wherein the three-dimensional structures are globular structures consisting of unilamellar bilayers.
